(19) United States
(12) Patent Application Publication
(10) Pub. No.: US 2003/0144879 A1
(43) Pub. Date: Jul. 31, 2003

(54) System for providing medical service

(76) Inventors: Shinji Hata, Tokyo (JP); Chikako Narita, Tokyo (JP)

Correspondence Address: Katten Muchin Zavis Rosenman, 15th Floor, 575 Madison Avenue, New York, NY, 10022-2585, US

(21) Appl. No.: 10/221,835

(22) PCT Filed: Feb. 26, 2002
(86) PCT No.: PCT/JP02/01724

(30) Foreign Application Priority Data
Feb. 28, 2001 (JP) 2001-54933
Feb. 26, 2002 (JP) 091902

(51) Int. Cl. G06F 17/60; G06F 19/00; G01N 33/48; G01N 33/50

(52) U.S. Cl. 705/2; 702/20

(57) ABSTRACT

A medical service providing system that provides medical service based on personal information of testee collects personal information on testee via electronic network, decides the best medical network for testee based on the collected personal information, and transmits information on the decided medical service to testee via electronic network. According to such configuration, unlike existing medical service providing system requiring plenty of people and expensive equipment, a substantial cut down in costs, labor and time for providing medical service based on the personal information from testee is realized.

### TECHNICAL FIELD

The present invention relates to a medical service providing system, a terminal device, a medical service providing method, a medical service providing program, and a computer readable recording medium having recorded therein a medical service providing program, all of which is suitably applicable to the processing for providing various medical services through the use of genetic information detected from testee.

### BACKGROUND ART

Following the rapid progress of recent scientific technology, various medical services made possible by collecting and analyzing information concerning DNA (Deoxyribo Nucleic Acid), a substance composing human genes, are becoming realized. Especially, technology to arrange and record DNA data collected from individuals via saliva and blood, etc. on DNA chips and other technology related to DNA chips have been researched extensively at many research institutions, leading to the rapid advance of this technical level in recent years.

Usually, collection of DNA data to be recorded on DNA chips is conducted by specialists. The data recorded on chips is analyzed and processed by specialized institutions such as hospitals and laboratories. Due to these circumstances, a tremendous amount of work and time are necessary until services using DNA data recorded on DNA chips become available to the public. Furthermore, since the equipment used for the collection and analyzing the DNA data is very expensive, it is extremely difficult to provide services using DNA data recorded on DNA chips to the public at a reasonable price.

As DNA data to be collected from individuals include information liable to change daily, it is necessary to carry out analyzing processing of DNA data on a daily basis. However, due to the fact that the work is done manually and the equipment is costly, it is difficult to carry out the data analysis on a daily basis. Therefore, conventional medical service providing systems cannot predict symptoms supposed to appear later on testee and reflect the predicted result on medical service on the basis of testee's DNA data that changes daily.

Though it is extremely important to ensure the safe and reliable management of DNA data, as the DNA data of each individual includes a huge amount of personal information and is therefore top personal information, there are presently no reliable information administrators or information processing systems that provide enough confidentiality and privacy. Therefore, in order to spread the medical service providing system and providing testee with medical service of good quality, it is desired that the technique enable of managing DNA data in high security is provided.

The present invention is done in view of the above-described problems, and its object is to provide a medical service providing system, a terminal device, a medical service providing method, a medical service providing program, and a computer readable recording medium having recorded therein a medical service providing program, capable of greatly reducing the costs, labor and time necessary for providing various medical services by use of genetic information detected from the testee.

### DISCLOSURE OF INVENTION

According to the present invention, the medical service providing system providing various medical services in accordance with genetic information detected from the testee, comprises of: means for collecting the testee's genetic information via the electronic network; and means for transmitting medical service information decided according to the collected genetic information via the electronic network.

In other words, the medical service providing system according to the present invention collects genetic information via the electronic network and transmits medical service information determined based on the collected genetic information via the electronic network. According to such a configuration, unlike conventional medical service providing system that needs much labor and costly equipment, it is possible to greatly reduce the costs, labor and time necessary in providing medical services based on genetic information detected from testee.

### BRIEF DESCRIPTION OF THE DRAWING

FIG. 1 is a diagram that shows composition of the medical service providing system according to an embodiment of the present invention;

FIG. 2 is a diagram that shows composition of DNA information collecting terminal device according to an embodiment of the present invention;

FIG. 3 is a flow chart that shows the operation of the medical service providing system according to an embodiment of the present invention;

FIG. 4 is a flow chart that shows the operation of the medical service providing system according to an embodiment of the present invention; and

FIG. 5 is a flow chart that shows the operation of the medical service providing system according to an embodiment of the present invention.

### BEST MODE FOR CARRYING OUT THE INVENTION

The medical service providing system according to the present invention is applicable and is able to be implemented, for example, towards a medical service providing system which uses DNA chips having recorded with DNA data. Throughout the following explanation, information on DNA collected from the user's saliva, blood, etc. shall be referred to as “DNA information”, and information concerning the base sequence of genes obtained through analysis of this DNA information will be referred to as “DNA data”.

Structure of the Medical Service Providing System

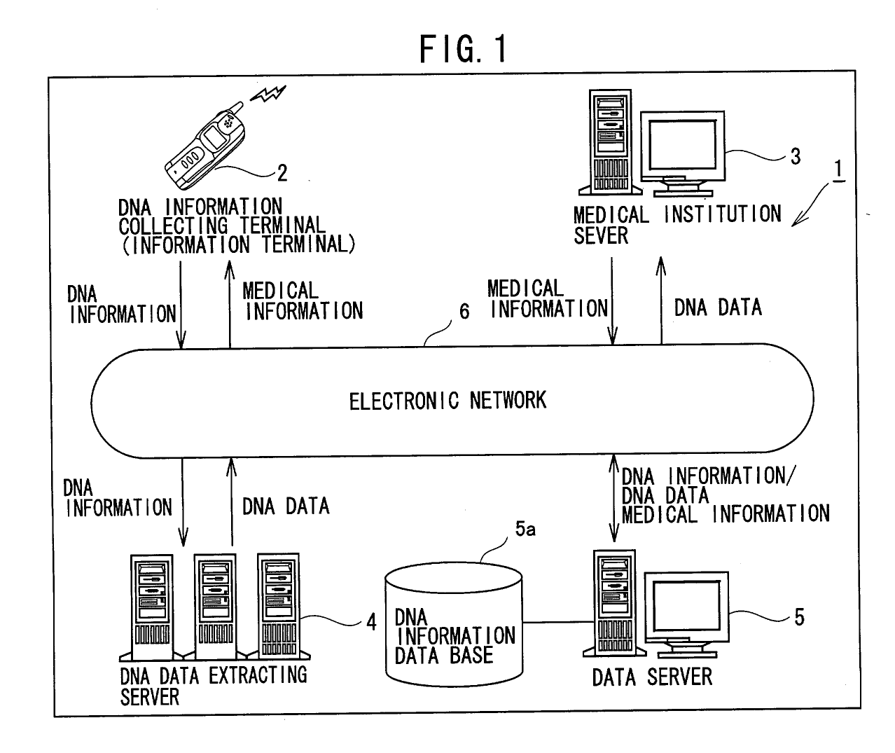

First, the structure of the medical service providing system according to an embodiment of the present invention is explained with reference to FIG. 1.

As shown in FIG. 1, the medical service providing system 1 according to an embodiment of the present invention comprises of: a DNA information collecting terminal (terminal device) 2, a medical institution server 3, a DNA data extracting server 4, and a data server 5. These devices are connected via an electronic network 6 to enable sending and receiving of data.

The electronic network 6 here refers to the entire communication network using electronic communications technology, including the use of telecommunication circuits, Internet systems based on Transmission Control Protocol/Internet Protocol (TCP/IP), Wide Area Network (WAN), Local Area Network (LAN), light fiber communications, cable communications, satellite communications, etc.

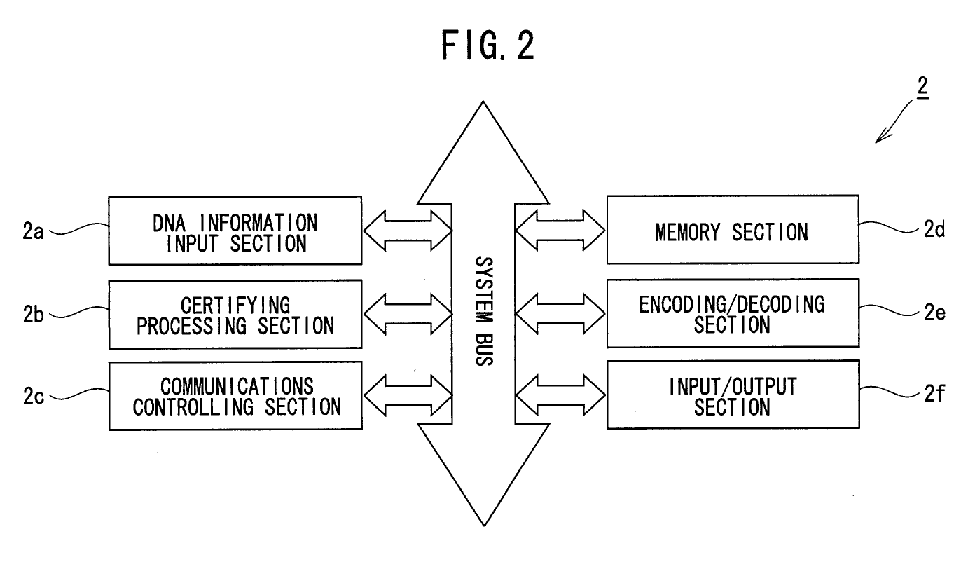

The DNA information collecting terminal 2 is provided on terminal equipment for information communication (e.g. PDA, Personal Data Assistant), and, as shown in FIG. 2, comprises of a DNA information input section 2a, a certifying processing section 2b, a communications controlling section 2c, a memory section 2d, an encoding/decoding section 2e, and an input/output section 2f. The DNA information collecting terminal 2 can be set at places to collect testee's DNA information easily, such as in tooth brushes, toilets and so on.

The DNA information input section 2a is the part where materials needed to detect and input DNA information of the user through samples such as saliva, blood, hair, skin, nail, excrement etc. are inputted. In the present embodiment, this DNA material input section 2a is detachable from the main body of DNA information collecting terminal 2 and can be exchanged each time DNA information is inputted, enabling acquisition of DNA information in a constantly fresh condition.

The certifying processing section 2b keeps certifying information of the user by utilizing high confidential method such as storing certifying information into IC chip. Also, the certifying processing section 2b controls the register process of DNA information, the production process of DNA data, and the certifying process during the analyzing process of DNA data, which shall later be referred to. The certifying processing section 2b may read the certifying information through the user's ID card, instead of storing it inside.

The communications controlling section 2c establishes a network connection with the electronic network 6 and sends data in a format according to the electronic network 6. The communications controlling section 2c converts data received through the electronic network 6 into a format processable by sections within the DNA information collecting terminal 2. Specifically, in the case the electronic network 6 is an analog line, the communications controlling section 2c is comprised of modems that modulate digital forms into analog forms and change received analog forms into digital forms.

Meanwhile, due to the huge amount of collected DNA information, it is desirable that the communications controlling section 2c adopts IPv6, one of the Internet Protocol, so as to assign IP address to each gene and send DNA information. According to this composition, the huge amount of DNA information can be managed based on the IP addresses assigned to each gene, and labor for managing the DNA information will be reduced.

The memory section 2d stores DNA information of the user inputted through the DNA information input section 2a. The encoding/decoding section 2e performs the encoding and decoding of the designated data according to the prescribed encoding and decoding keys. When an encrypting technology for quantum is applied to encoding/decoding process for designated DNA information, it will avoid improper encoding/decoding of data and make possible to keep data with high confidentiality.

The input/output section 2f performs the input/output process of various information related to medical service providing. Various methods such as keyboard methods, touch panel methods, ten-key methods, mouse pointer methods, light pen methods are applicable as input forms. On the other hand, display methods, printer methods, sound methods, light methods, etc. are methods applicable as output forms.

The data server 5 objectifies DNA information sent from DNA information collecting terminal 2. The data server 5 files the objectified DNA information by assigning unique reference keys of each user and stores the filed DNA information into DNA information database 5a. This data server 5 possesses identification information utilized in the following user certification processing.

The DNA data extracting server 4 extracts DNA data from DNA information file stored in DNA information database 5a and records the extracted DNA data on DNA chip.

The medical institution server 3 analyzes the user's health condition based on the DNA data that the DNA data extracting server 4 extracted and then determines the best treatment instructions for the user (=medical service information) according to the results of the analysis result.

Operation of the Medical Service Providing System

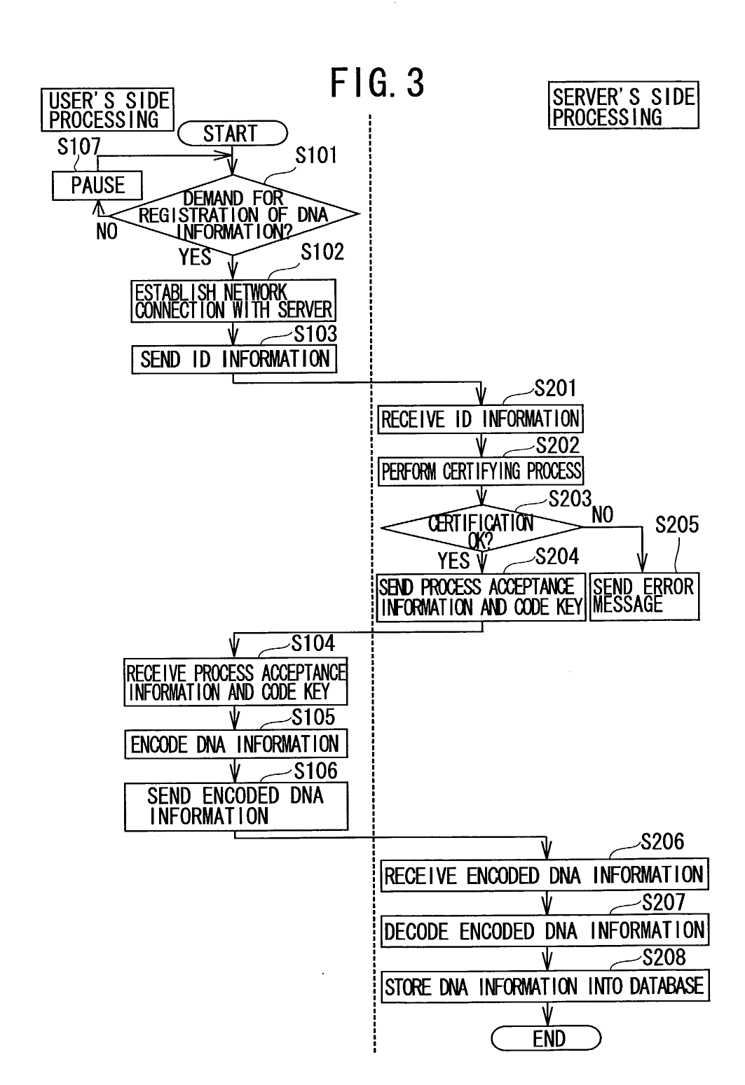

Next, in reference to FIG. 3-5, the operation of the medical service providing system is explained dividing to the three processings: (1) registration process of DNA information, (2) generating process of DNA data, and (3) analyzing process of DNA data.

Registration Process of DNA Information

Next, the register process of DNA information is explained in reference to the flow chart shown in FIG. 3.

Within the medical service providing system 1 according to the present embodiment, after user records his/her DNA information into memory section 2d by operating DNA information input section 2a; inserts his/her ID card into certifying processing section 2b of the DNA information collecting terminal 2; and presses the DNA information registration demand button by operating input/output section 2f, the register process of DNA information starts.

In accordance to the user's pressing of the DNA information registration demand button, the communications controlling section 2c of the DNA information collecting terminal 2 establishes a network connection between the DNA information collecting terminal 2 and the data server 5 (S102). Once a network connection is established between the DNA information collecting terminal 2 and the data server 5, the certifying processing section 2b reads out the certifying information of the user (hereafter referred to as “ID information”) from ID card, and sends via electronic network 6 the ID information of the user who requests registration of his/her DNA information towards the data server 5 (S103)

The ID information sent to the data server 5 refers mainly to the user's attributions such as the user's name, password, date of birth, however, it is also possible to apply other information such as the device number or telephone number that is uniquely applied to DNA information collecting terminal 2 and perform the following certifying process by utilizing these information.

After receiving the user's ID information from the DNA information collecting terminal 2 (S201), the data server 5 determines whether the ID information stored on the data server's side 5 and the ID information sent through the electronic network 6 correspond, to thereby perform the certifying process (S202).

In order to carry out the certifying process through the electronic network 6, it is desirable to have the user register the necessary information for certification within the data server 5 beforehand. Yet it is also possible to have the user registration done during the registration process of DNA information.

Once the user sending the registration demand of DNA information is authorized as the result of the certifying process, the data server 5 sends to the DNA information collecting terminal 2 the process acceptance information that accepts the registration operation of DNA information and a code key to encode the DNA information (S204) . On the other hand, if the authorizing fails, the data server 5 sends to the DNA information collecting terminal 2 an error message such as “This ID information is invalid. Please reenter the correct ID information” (S205).

When the process acceptance information and code key sent from the data server 5 are received at the DNA information collecting terminal 2 (S104), the encoding/decoding section 2e encodes the DNA information stored within the memory section 2d, using the received code key (S105). Then the communications controlling section 2c sends the encoded DNA information to the data server 5 (S106).

After receiving the encoded DNA information from the DNA information collecting terminal 2 (S206), the data server 5 decodes the encoded DNA information (S207) . After object (meta-data) conversion of the decoded DNA information, the data server 5 converts the DNA information to file format by adding search keys to each DNA attribute information in reference to the IP address assigned to each DNA, and stores the file-formatted DNA information to the DNA information database 5a (S208). By managing the DNA information in the database on the basis of the IP address assigned to each DNA as mentioned above, personal information will be protected and controlled efficiently as well as effectively.

Data server 5 may store the DNA information file in DNA information database 5a after encrypting the DNA information file, to thereby realize high confidentiality for DNA information. In this case, it is desirable to assign IP address to each encrypted DNA information file and manage the encrypted DNA information by utilizing the assigned IP address. The IP address assigned to the encrypted DNA information file may be encrypted so as to realize high confidentiality.

Generating Process of DNA Data

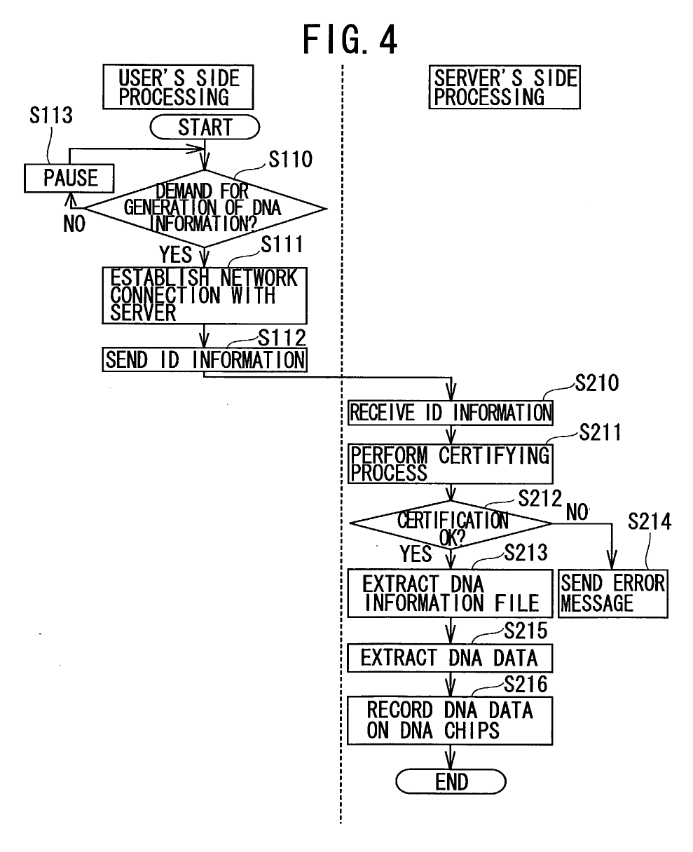

Next, the process of generating DNA data from registered DNA information is explained in reference to the flow chart shown in FIG. 4.

Within the medical service providing system 1 according to the present embodiment, when the user presses the DNA data generating demand button through the input/output section 2f after inserting his/her ID card through the certifying process section 2b of the DNA information collecting terminal 2, the communications controlling section 2c of the DNA information collecting terminal 2 establishes a network connection between the DNA information collecting terminal 2 and the data server 5 (S111). When a network connection has been established between the DNA information collecting terminal 2 and the data server 5, the certifying processing section 2b sends the ID information of the user requesting the generating of DNA data through the electronic network 6 using the communications controlling section 2c (S112).

After receiving the user's ID information from the DNA information collecting terminal 2 (S210), the data server 5 determines whether the ID information stored on the data server 5 side and the ID information sent through the electronic network 6 correspond, to thereby perform the certifying process (S211).

Once the user who has sent the registration demand of DNA information is authorized as result of the certifying process, the data server 5 extracts the DNA information file corresponding to the authorized user from the DNA information database 5a in reference to the search keys, and then sends the extracted DNA information file to the DNA data extracting server 4 through the electronic network 6 (S213).

In the present embodiment, when sending the DNA information file towards the DNA data extracting server 4, the data server 5 encrypts the DNA information so as to preventing the data from being browsed or being made alterations. Further, the data server 5 attaches an electronic “watermark” to the DNA information file. According to this electronic “watermark”, even in case a third person with ill intention made alterations unfairly to the DNA information file, it is possible to detect such action and judge that the sent DNA information is injustice. The data file format of DNA information file is constituted from the language both the data server 5 and the DNA data extracting server 4 can process such as Hyper Text Markup Language (HTML) or Extensible Markup Language (XML), and is recommended to be of a structure with inter-operability.

On the other hand, if the authorizing fails, the data server 5 sends to the DNA information collecting terminal 2 an error message such as “This ID information is invalid. Please reenter the correct ID information” (S214).

Once receiving the DNA information file from the data server 5, the DNA data-extracting server 4 decodes the received DNA information file after the certifying procedure. Then the DNA data-extracting server 4 extracts DNA data from the decoded DNA information file (S215) and records the extracted DNA data on to DNA chips (S216).

It is also possible for the DNA data-extracting server 4 to send the extracted DNA data to the data server 5 through the electronic network 6, and record the DNA data on DNA chips on the data server 5 side. In this case, as the DNA data on DNA chips will be stored and managed within the DNA information database 5a of the data server 5, it is possible to enhance the security of personal information.

The process of extracting DNA data from DNA information and recording the extracted DNA data on to DNA chips will follow the generally known techniques, however, the following procedure is recommended:

1. Extract RNA from the DNA information.

2. Color RNA by chemical or enzymatic treatment, express the four base sequences by color, and read out these optically.

3. Record this base sequence data on the DNA chips or rewrite the existing records on the DNA chips.

RNA here refers to ribonucleic acid structured from nucleotide, which consists of sugar, phosphoric acid, and bases like DNA. However, while deoxyribose is the sugar for DNA, the sugar for RNA is ribose. Furthermore, while the three bases A, G, C among the four bases are common with DNA, the base U (Uracil) is the substance facilitating the generation of proteins instead of the base T in DNA. This base U is divided into three types according to function as follows: mRNA (messenger RNA), tRNA (transfer RNA), and rRNA (ribosome RNA).

The above example considered information content from the four colored base sequence as DNA data. However, if it is possible to chemically treat the bases' color sequence/tone sequence by file-formatting the base sequence represented in tones, DNA data can be classified according to the size of DNA pieces, attached to a ‘gene expression profile’ of a specific cell, and can be used for treatment hereafter.

Furthermore, information on the four colored base sequence with individual IP address shall be stored inside DNA information database 5a. When analyzing base sequence, an analyst will collect information on the four colored base sequence from DNA information data case 5a according to the IP address and rearranges the base sequence. Through such procedure, it is possible to keep the information on base sequence with high confidentiality.

Analyzing Process of DNA Data

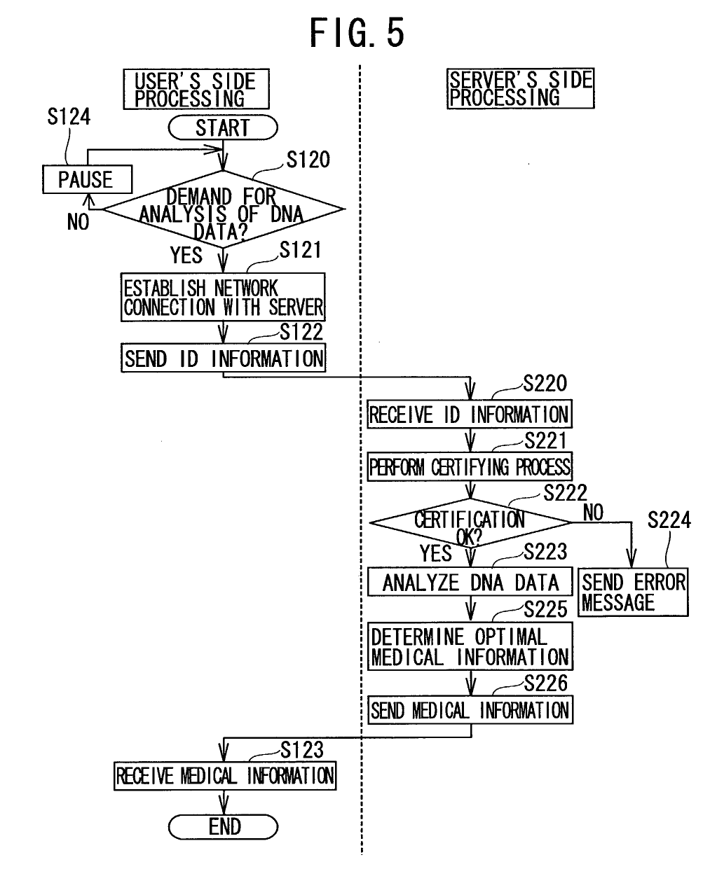

Finally, the process of determining the medical service information to be sent to the user based on their DNA is explained in reference to the flow chart shown in FIG. 5.

Within the medical service providing system 1 according to the present embodiment, when the user presses the DNA data analysis demand button through the input/output section 2f after inserting his/her ID card through the certifying processing section 2b of the DNA information collecting terminal 2, the communications controlling section 2c of the DNA information collecting terminal 2 establishes a network connection between the DNA information collecting terminal 2 and the data server 5 (S121). When a network connection has been established between the DNA information collecting terminal 2 and the data server 5, the certifying processing section 2b sends the ID information of the user requesting the generation of DNA data through the electronic network 6 using the communications controlling section 2c (S122).

Meanwhile, during the process of the above-mentioned step S122, DNA information collecting terminal 2 may be set to send the user's information related to providing medical service. The information shall be inputted by the user beforehand, the information including his/her will to be an organ donor, to be a monitor for new drag, or to choose Western medicine medical service or Eastern medicine medical service. If the information is sent together, data server 5 and medical institution server 3 will be able to provide medical service which suits the testee's will and be able to utilize the information sent from the testee for improving quality of medical service to other testee as well.

After receiving the user's ID information from the DNA information collecting terminal 2 (S220), the data server 5 determines whether the ID information stored on the data server 5 side and the ID information sent through the electronic network 6 correspond, to thereby perform the certifying process (S221).

Once the user who has sent the registration demand of DNA information is authorized as the result of the certifying process, the data server 5 encodes DNA data on the DNA chips, sends the encoded DNA data to the medical institution server 3 through the electronic network, and performs the following procedures.

Even during the sending of DNA data, a mutual certifying procedure is performed between the data server 5 and the medical institution server 3 in order to ensure security. On the other hand, if the authorizing fails, the data server 5 sends to the DNA information collecting terminal 2 an error message such as “This ID information is invalid. Please reenter the correct ID information” (S224).

After receiving the encoded DNA data from the data server 5, the medical institution server 3 decodes the encoded DNA data and analyzes the user's health condition according to the decoded DNA data (S223).

It is desirable to utilize the following technologies as the analyzing method for DNA data: photography, X rays, and diagnostic process with images generated by computer graphics. And, followed by analysis on mutations and damages of DNA, the present condition of the user can be predicted.

After analyzing health condition of the user, the medical institution server 3 determines the information concerning the necessary (optimal) therapy for the user (=medical service information) (S225), and sends the medical service information either directly or through the data server 5 towards the DNA information collecting terminal 2 (S226). It is desirable that the medical service information is transmitted towards the DNA information collecting terminal 2 in encoded form so as to enhance security during its sending. When the medical service information has been sent from the medical institution server 3, the user receives this medical service information through the DNA information collecting terminal 2 (S213) and performs the suitable medical treatment according to the medical service information.

As mentioned above, according to the medical service system of the present embodiment, information related to DNA data is sent and received entirely on the electronic network 6, it is possible to configure an information processing system related to DNA information at a low cost.

In addition, since this enables users to send their DNA information at a low cost on a daily basis, it is also possible to keep a chronological survey of the DNA information, leading to a further advanced medical analysis at low cost and shorter time compared to other conventional methods. Further, it is possible to predict symptoms supposed to develop on user in the future on the basis of user's DNA data that varies daily and contribute to prevent a disease.

Furthermore, as data is encoded using the code key provided by the data server 5 when sending DNA data related information, unfair use or leakage of DNA data is prevented, and confidentiality and secrecy is enhanced.

The present invention carried out by the inventor has been explained so far, but the explanation and figures that constitute part of this disclosure should not be understood as restriction of the present invention. This disclosure should present clearly towards the enterpriser various alternate embodiments, examples of execution and practicing techniques.

For example, the data server 5 which collects DNA information, the DNA data-extracting server 4 which generates DNA data from DNA information, the medical institution server 3 which determines the optimal medical service information for the user based on the generated DNA data are structured separately within the implementing form described so far, but it would be entirely acceptable to unify these structures into one system.

Also, the processing within the above-mentioned servers and the DNA information collecting terminal 2 may be programmed respectively and saved into a computer readable recording medium. Upon executing each process, the processing of the above implementing form could be realized by reading out this recording medium on to a computer system, storing the program into the memory section of the computer system, and performing the program by an operating device. Recording mediums as mentioned here refer to computer readable recording mediums that are capable of recording programs such as semiconductor memories, magnetic disks, laser disks, laser magnetic disks, and magnetic tapes.

While the DNA information itself was sent through the electronic network in the present embodiment, it is also acceptable to carry out the analyzing process on the side of the DNA information collecting terminal 2, and for example, send information on RNA and base sequence through the electronic network. Also, while DNA information is to be collected via information collecting terminal 2 in a portable form, it can take any form if the information collecting terminal 2 is able to collect DNA information. For instance, the composing section of information collecting terminal 2 can be attached with following equipment: portable information communication terminal, such as mobile phones; tooth brushes; fixed equipment, such as toilets and so on.

Furthermore, information collecting terminal 2 is also acceptable to collect and send the user's information on information transmitting material, such as proteins, hormones, amino acids, oxygen and so on. For instance, when information related to proteins is to be collected, a medical service utilized for preventing the user's disease will be realized. Because it comes to be known recently that an analysis on function and three-dimensional composition of proteins will indicate pathogenic mechanisms.

In the meantime, medical service was offered via DNA chips in the present embodiment, the following forms are also acceptable to record the user's living information and provide the medical service besides DNA chips: RNA chips, protein chips, neuron chips and hormone chips.

Furthermore, while the user's DNA information and DNA data were stored directly within the DNA information database 5a in the present embodiment, it would also be acceptable to, for example, provide a standardized data for DNA information and DNA data within the DNA information database 5a, and in storing the users' DNA information and DNA data, extract the difference between the stored data and template data, and store the difference data into the DNA information database for each users. According to such structure, it is possible to reduce the burden on the database as well as simplify the users' medical diagnosis through effective use of the template data.

Further, in the present embodiment, while the user's certifying process was effected each time the registration process of the DNA information, the generating process of the DNA data and the analyzing process of the DNA data were carried out, it would of course be acceptable to perform the entire procedures sequentially in one certifying process as long as security is maintained.

It is also desirable for the medical institution server 3 to file the health data of each user (=individual health files) or generate medical recipes according to each user's health characteristic by utilizing the DNA data received several times in chronological order. This enables the medical institution server to promptly determine the optimal medical service information towards the user based on the generated individual health files or medical recipes. In this case, it is desirable that the data server 5 converts the user's health file and medical recipe into a database and is responsible for its storage and management.

Additionally, upon converting the DNA chip information into a database, the data server 5 may set up a parameter for the individual health data file, and generate and manage a database suiting individual demands. According to such structure, it would be possible to distribute health information corresponding to the individual users by using information stored within the database, and medical recipes obtained from medical institutions could be sent to the users as well. Furthermore, as unnecessary data for the users can be deleted, the burden of the data server 5 could be reduced. It is also possible for the data server 5 to set up parameters for health information data for each user, generate health information data for users, and store the generated data within the database.

Further, the data server 5 may send the stored health information towards a designated health insurance company according to instructions from the user. According to such structure, health insurance companies may arrange for contracts requested by the user such as health insurance, injury insurance, etc. based on information sent from the data server 5. It could also support the production of health insurance menus and catalogues. In this case, the data produced by health insurance companies is sent to the database of the data server 5 through the electronic network and will be stored and managed. The data server 5 may also use this data as health information for each user at the request of the users.

As seen above, it should be understood that this invention includes various embodiments not mentioned here. Therefore, this invention should be limited only by invention specific items concerning the appropriate extent of claims from this disclosure.

### Industrial Applicability

The present invention is applicable to medical service providing system, which uses DNA chips with recorded DNA data.

1. A medical service providing system comprising:
means for transmitting personal information of testee through electronic network, provided on the testee side;
means for determining optical medical service for the testee based on the transmitted personal information of testee, provided on medical service provider side; and
means for transmitting information on the determined medical service through electronic network, provided on the medical service provider side.

2. A medical service providing system according to claim 1, wherein the personal information is the genetic information of the testee.

3. A medical service providing system comprising:
genetic information transmitting means for transmitting genetic information of testee through electronic network, provided on the testee side;
medical service determining means for determining optical medical service for the testee based on the transmitted genetic information, provided on medical service provider side; and
medical service transmitting means for transmitting information on the determined medical service through electronic network, provided on the medical service provider side.

4. A medical service providing system according to claim 3, wherein the genetic information transmitting means transmits the genetic information in encoded form, and the medical service transmitting means transmits the information on the medical service in encoded form.

5. A medical service providing system according to claim 3, further comprising:
storage means for storing the transmitted genetic information and information on the medical service corresponds to the genetic information for each testee, provided on the medical service provider side,
wherein the medical service determining means determines optical medical service for the testee by referencing the information stored in the storage means.

6. A medical service providing system according to claim 5, wherein the storage means stores the transmitted genetic information with assigning unique IP address, and the medical service determining means reads out the genetic information from the storage means utilizing the IP address so as to determine optical medical service.

7. A medical service providing system according to claim 3, wherein the genetic information includes at least one of DNA information and protein information of the testee.

8. A medical service providing system according to claim 5, wherein the storage means stores information on the medical service the testee requests.

9. A medical service providing system according to claim 5, wherein the information stored in the storage means is configurable to keep public or private by the testee.

10. A medical service providing system according to claim 3, wherein the electronic network includes at least a wireless communication network.

11. A terminal device comprising:
detecting unit for detecting genetic information of testee; and
transmitting unit for transmitting the detected genetic information through electronic network.

12. A terminal device according to claim 11, further comprising:
receiving unit for receiving information on medical service for the testee, the medical service determined based on the genetic information.

13. A terminal device according to claim 11, further comprising:
encoding unit for encoding the genetic information,
wherein the transmitting unit transmits the genetic information encoded by the encoding unit.

14. A terminal device according to claim 10, further comprising:
certifying processing unit for controlling the processing of transmitting the genetic information through electronic network,
wherein the certifying processing unit enables the processing of transmitting the genetic information through electronic network only for the authorized testee.

15. A medical service providing method comprising the steps of:
transmitting genetic information detected from testee through electronic network;
determining optical medical service for the testee based on the transmitted genetic information; and
transmitting information on the determined medical service through electronic network, provided on the medical service provider side.

16. A medical service providing method according to claim 15, further comprising the steps of:
storing the transmitted genetic information with assigning unique IP address; and
reading out the genetic information by specifying the IP address so as to determine optical medical service for the testee based on the read-out genetic information.

17. A medical service providing method according to claim 15, wherein the genetic information includes at least one of DNA information and proteins information of the testee.

18. A medical service providing method according to claim 15, further comprising the step of:
encoding the genetic information and the information on the medical service that is transmitted through electronic network.

19. A medical service providing program to be executed on a computer, comprising the steps of:
transmitting genetic information detected from testee through electronic network;
determining optical medical service for the testee based on the transmitted genetic information; and
transmitting information on the determined medical service through electronic network, provided on the medical service provider side.

20. A computer readable recording medium having recorded therein a medical service providing program to be executed on a computer, wherein the medical service providing program comprising the steps of:
transmitting genetic information detected from testee through electronic network;
determining optical medical service for the testee based on the transmitted genetic information; and
transmitting information on the determined medical service through electronic network, provided on the medical service provider side.
